(19) United States
(12) Patent Application Publication
(10) Pub. No.: US 2004/0229336 A1
(43) Pub. Date: Nov. 18, 2004

(54) Method for in vitro culture of viruses of the togaviridae and flaviviridae families and uses

(75) Inventors: Patrice Andre, Lyon (FR); Vincent Lotteau, Vourles (FR); Glaucia Paranhos-Baccala, Lyon (FR); Florence Komurian-Pradel, Poleymieux (FR)

Correspondence Address: OLIFF & BERRIDGE, PLC, P.O. BOX 19928, ALEXANDRIA, VA, 22320, US

(73) Assignees: Bio Merieux, Marcy L'Etoile (FR); Institut National De La Sante Et De La Recherche Medicale, Paris (FR)

(21) Appl. No.: 10/776,617

(22) Filed: Feb. 12, 2004

Related U.S. Application Data
(63) Continuation of application No. 10/031,439, filed on Feb. 25, 2002, now abandoned.
Application No. 10/031,439 is a 371 of international application No. PCT/FR00/02202, filed on Jul. 31, 2000.

(30) Foreign Application Priority Data
Jul. 30, 1999 (FR) 99 10095

(51) Int. Cl. C12N 7/00

(52) U.S. Cl. 435/235.1

(57) ABSTRACT

The invention concerns a method of culturing, propagating and replicating in vitro viruses of the Togaviridae and Flaviviridae families which consists in: providing at least a fraction of LVP's obtained from serum or plasma of a patient infected with a least a virus of the Togaviridae and Flaviviridae families; contacting said fraction, for a predetermined time interval in an appropriate culture medium, with permissive cells having an endocytosis path relayed by at least one receptor of the lipoproteins and modulated inter alia by an activating agent selected among an unsaturated fatty acid or an unsaturated fatty acid derivative comprising 16 to 20 carbon atoms or their mixture. The invention also concerns the uses of viral particles and polypeptides obtained by said method.

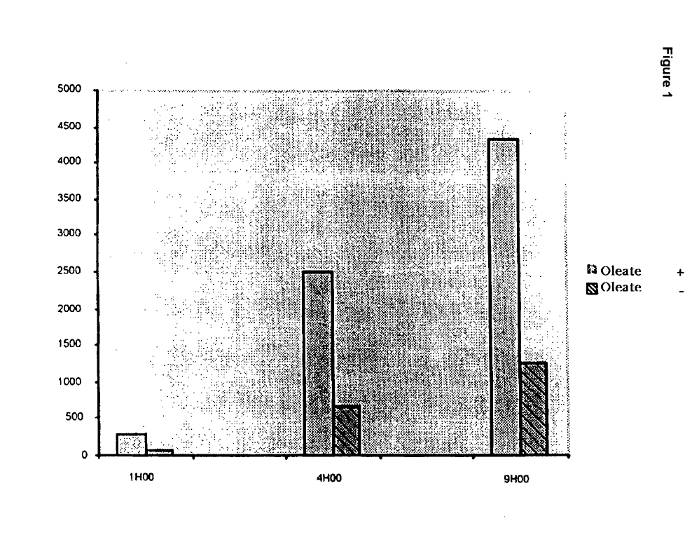

This is a Continuation of application Ser. No. 10/031,439, which is a National Stage Application of PCT/FR00/02202 filed Jul. 31, 2000. The entire disclosure of the prior applications is hereby incorporated by reference herein in its entirety.

The present invention in particular relates to a method for culturing and propagating in vitro, an RNA virus involved in the development of human viral pathologies.

The culturing and propagating method of the invention applies mainly to viruses belonging to the Togaviridae and Flaviviridae families. These viruses have the common characteristics of being enveloped, non-segmented, positive-sense, single-stranded RNA viruses.

The Togaviridae family comprises the viruses of the Alphavirus and Rubivirus genera. The virions are 70 nm in diameter, are spherical and comprise an envelope and peplomers composed of a heterodimer of two glycoproteins. The genome consists of a single molecule of single-stranded RNA of 9.7 to 11.8 kb. The structural proteins include a protein of the capsid and two envelope glycoproteins. The virions contain lipids derived from the hosts' cell membrane. Replication involves the synthesis of a complementary RNA strand which is used as a matrix for the synthesis of the genomic RNA. The genomic RNA is used as a matrix for an intermediate replication RNA, which, in turn, is used as a matrix for the synthesis of mRNA and the production of a polyprotein precursor which is cleaved in order to obtain structural and nonstructural proteins. Replication takes place in the cytoplasm and assembly involves budding through the membrane.

The Sindbis virus, the equine encephalitis viruses (western, eastern, Venezuelan), the chikungunya virus, the o'nyong-nyong virus, the Igbo Ora virus, the Ross River virus, the Mayaro virus and the Barmah Forest virus belong to the Alphavirus genus and are agents which are pathogenic to humans. The hepatitis E virus is currently classified in the Alphavirus group, although it is nonenveloped and smaller in size. The rubella virus, which is classified in the Rubivirus genus is also an agent which is pathogenic for humans.

Classified in the Flaviviridae family are the viruses of the Flavivirus (flaviviruses), Pestivurus (mucosal disease viruses) and Hepacivirus (hepatitis C and G viruses) genera. The virions are spherical with a diameter of 45 to 60 nm in diameter and consist of a lipid bilayer enveloping an icosahedral capsid enclosing the genome. The genome consists of a single molecule of positive-sense, single-stranded, linear RNA of 10.7 kb for the flaviviruses, 12.5 kb for the pestiviruses and 9.5 kb for the hepatitis C virus. The virions contain two or three membrane-associated proteins and one core protein. The virions contain lipids derived from the host's cell membranes. They contain carbohydrates in the form of glycolipids and glycoproteins. Replication involves the synthesis of the complementary RNA which is used as a matrix for the synthesis of the genomic RNA. A single reading frame (ORF) encodes a polyprotein which is cleaved by viral and cellular proteolysis. The structural proteins are encoded by the 5′ end and the nonstructural proteins are encoded by the 3′ end of the RNA. Replication takes place in the cytoplasm and assembly involves passing through and envelopment by the membranes of the host's endoplasmic reticulum. Replication is accompanied by a characteristic aspect of intracellular membrane proliferation.

The yellow fever virus, the dengue virus, the Japanese encephalitis virus, the West Nile virus, the St. Louis encephalitis virus, the Murray Valley virus, the tick-transmitted encephalitis viruses and the Israel Turkey meningoencephalitis virus belong to the Flavivirus genus and are agents which are pathogenic for humans.

The bovine diarrhoea virus, the hog cholera virus and the sheep border disease virus belong to the Pestivurus genus.

With regard to the Hepacivirus genus, it currently contains the hepatitis C and hepatitis G viruses (GBV-C and the GBV-A and GBV-B viruses) which are well known for being agents which infect humans.

Specifically, hepatitis C is the main cause of hepatitis acquired by transfusion. Hepatitis C can also be transmitted via other percutaneous routes, for example by intravenous injection of drugs. The risk of contamination of health professionals is, moreover, not insignificant.

Hepatitis C differs from the other forms of liver diseases associated with viruses, such as hepatitis A, B or D. Infections with the hepatitis C virus (HCV or VHC) are often chronic resulting in liver diseases, such as hepatitis, cirrhosis and carcinoma, in a large number of cases.

Although the risk of transmission of the virus by transfusion has decreased due to blood donors being selected, the frequency of hepatitis C remains high. Currently, approximately 170 million people throughout the world are chronically infected with HCV. The populations at high risk are mainly those who have received transfusions and intravenous drug users, but asymptomatic blood donors exist who do not belong to these high risk groups and in whom circulating anti-HCV antibodies have been found. For the latter, the route of infection has not yet been identified.

HCV was the first hepatotropic virus isolated using molecular biology techniques. The sequences of the viral genome were cloned before the viral particle was visualized.

HCV is a positive single-stranded RNA virus, of 9.5 kb, which replicates via a complementary RNA copy, the translation product of which is a precursor of a single polyprotein of approximately 3000 amino acids. The 5′ end of the HCV genome corresponds to an untranslated region adjacent to the genes which encode the structural proteins, the core protein of the nucleocapsid and the two envelope glycoproteins, E1 and E2/NS1. The 5′ untranslated region and the core gene are relatively well conserved in the various gene types, but the E2 envelope proteins are encoded by a hypervariable region which is different form one isolate to another. The 3′ end of the HCV genome contains the genes which encode the nonstructural (NS) proteins and a well-conserved 3′ noncoding region.

Because of its genomic organisation and its presumed replication method, HCV has been classified in a new genus of the Flaviviridae family, the Hepaciviruses.

Many techniques have been developed for diagnosing and HCV infection. For example, diagnostic immunoassays have been carried out to detect antibodies directed against HCV proteins in the sera of patients. Synthesis of cDNA by reverse transcription of the virus RNA and amplification by PCR have also been used to detect the HCV genome, such as the indirect measurement of a potentially infectious virus in the sera of chronically infected humans or those of experimentally infected chimpanzees. Furthermore, based on gene cloning, DNA-probe hybridization techniques have also been developed.

However, it is acknowledged that the existing diagnostic techniques lack sensitivity and/or specificity, and/or suffer from implementation difficulties. By way of example, with the probe hybridization method, it is impossible to distinguish between a virus with low infectious power and a virus with high infectious power. It is therefore necessary, but this is difficult to carry out, to inoculate a chimpanzee with the virus which must be tested, and to test the resulting infection on the animal.

It is therefore of the utmost importance, from a public health point of view, to be able to develop specific, sensitive and practical methods for identifying and screening HCV carriers. One of the solutions may be to produce a very efficient in vitro culturing system for HCV, which would make it possible to obtain propagation of the virus, in particular for studying its replication mechanisms, for testing neutralizing or antiviral antibodies, and also for developing biological materials, diagnostic tests and vaccine preparations. In fact, although the complete HCV sequence has been available since 1989 (Q. L. Choo et al., Science 244, 359, (1989)), understanding of the life cycle and of the method of replication of HCV has been hampered by the lack of a suitable in vitro culturing system. Ito et al. (J. gen. Virol. 77: 1043-1054 (1996)) have indeed confirmed that HCV replication is maintained in primary cultures of human hepatocytes obtained from patients carrying HCV and for whom the disease was chronically established, and suggested a passage of infection, but problems relating to the propagation of the virus remain (impossible to culture long term) and the system developed is limited by the need to supply with human liver and the workload of the technique. Moreover, to date, there is no general consensus regarding the tropism of HCV, and not all the cellular receptors for the virus have yet been identified, in particular the receptors allowing endocytosis of the virus (Pileri et al., 1998, Science, 282, pages 938-941).

HGV is a virus which was discovered independently by two different teams, one named it HGV and the other GBV-C. It has a genomic structure close to that of the flaviviruses. Its 2900 amino acid polyprotein is encoded by a 9,400 nucleotide RNA, the 5′ end of which encodes structural proteins (truncated nucleocapsid and envelope) and the 3′ end of which encodes proteins which have a role in replication. It is transmitted by blood transfusion and can be detected in patients who have acute, chronic and violent forms of hepatitis. Its contribution in acute and chronic liver diseases is still poorly understood. GBV-A and GBV-B have also been described and are very close to HGV.

Particles containing viral RNA, very heterogeneous in density, are found in the plasma of patients infected with HCV. This heterogeneity in density of the particles containing viral RNA is attributed to their association in variable proportion with lipoproteins (Thomsen et al., 1993, Med. Microbiol. Immunol. 182: 639). In the description of the present patent application, the inventors have named these hybrid particles LVPs (lipo-viro-particles). The distribution of each of these forms along a density gradient varies from one patient to the other. Existing analyses of low density particles show densities covering those of the LDLs (low density lipoproteins) and of the VLDLs (very low density lipoproteins). The size described (50 nm) makes them close to the VLDLs. Moreover, the particles may be precipitated (sometimes even in their entirety, by anti-β-lipoprotein sera (Thomsen et al., 1992, Med. Microbiol. Immunol. 181:293; Prince et al., 1996, J. Viral. Hepat. 3:11).

It has also been observed that the hepatitis G virus may be precipitated, virtually in its entirety, by anti-β-lipoprotein sera obtained from patients chronically infected with HGV (Sato et al. Biochem. Biophys. Res. Commun. 229:719).

It has, moreover, been observed that infection of human hepatocytes, with HCV, is accompanied by intracytoplasmic accumulation of lipid vesicles. Transfection of the HCV gene encoding the capsid into a continuous line of human hepatocytes (Hep/G2) induces the same phenomenon (Barba et al., 1997, PNAS 94:1200). These vesicles are rich in triglycerides. The HCV capsid protein is arranged side by side at the surface of the vesicles, as is the apolipoprotein apo A-II.

The apolipoproteins allow lipids to be transported by introducing structural stability and solubility to the lipoprotein particles with which they are associated. They also determine the outcome for these particles within the metabolism, in particular by targeting them onto receptors.

Apo B 100 is the main apolipoprotein of VLDLs, LDLs and IDLs (intermediate density lipoproteins). It is synthesized in the liver and is essential for the assembly and secretion of VLDLs from the liver. It also constitutes a ligand for binding LDLs to their receptors. One of the receptors for LDLs, the LDL receptor, is a cell surface protein which binds and internalizes the lipoproteins rich in cholesterol which contain apo B 100 and apo E. Apo E is mainly synthesized in hepatocytes. It constitutes a ligand for binding various lipoproteins to the receptor for LDLs and to the LRP (LDL-receptor-related protein).

Apo B 48 is essential for the assembly and secretion of chylomicrons. It is encoded by the same gene and the same mRNA as apo B 100, but in the intestine, a mutation introduces a stop codon such that apo B 48 contains only 40% of the total length of apo B 100. Its role in the metabolism of chylomicrons in the plasma is poorly understood, but the individuals who exhibit mutations which interfere with its normal synthesis do not have any, or have very low levels of, chylomicrons, VLDLs, IDLs and LDLs.

The nature of the abovementioned LVPs containing viral RNA is not, to date, known, but the present inventors have put forward the hypothesis and verified that at least one of the cell surface receptors for lipoproteins contributes to the infection and propagation of viruses belonging to the Togaviridae and Flaviviridae families, in a cell culture. They have, in fact, shown that the LVPs are ligands for a receptor-associated endocytosis pathway, preferentially for the LSR (lipolysis-stimulated receptor), (Frances T. Yen et al., Biochemistry, 1994, 33, 1172-1180 and Bernard E. Bihain and Frances T. Yen, Biochemistry, 1992, 31, 4628-4636). They have, moreover, shown that the LVPs may be associated with human immunoglobulins.

On this basis, they have developed novel methods which allow the culturing, propagating and replicating, in vitro, of viruses belonging to the Togaviridae and Flaviviridae families. These methods, which make it possible to obtain propagation of these viruses, are useful in particular for studying their mechanisms of replication, for testing neutralising and antiviral antibodies, and for developing biological materials for diagnosis and therapy. Moreover, the methods of the invention make it possible to obtain an infected cell line which is useful for screening and/or selecting at least one antiviral molecule, by bringing the infected cell line and the antiviral molecule into contact.

According to this method, there is at least one LVP fraction or at least one LVP fraction associated with the human immunoglobulins, obtained from serum or from plasma of the patient infected with a virus belonging to the Togaviridae or Flavivirida family, and said LVP fraction is brought into contact, for a predetermined period of time in a suitable culture medium, with permissible cells which have an endocytosis pathway relayed by at least one receptor for lipoproteins and modulated by an activating agent chosen from unsaturated fatty acids, derivatives of unsaturated fatty acids comprising from 16 to 20 carbon atoms and mixtures thereof.

The receptor for lipoproteins is the LSR and/or the surface receptor for LDLs, and advantageously said permissive cells have both receptors.

The unsaturated fatty acid is chosen from oleic acid, palmitoleic acid, linoleic acid, linolenic acid, arachidonic acid, transhexadecenoic acid and elaidic acid, or derivatives thereof. In a preferred embodiment of the invention, the fatty acid selected is oleic acid, which is added to the culture medium at a concentration of between 0.1 and 1 mM, preferably 0.5 mM.

The cells are preferably cells chosen from human or animal hapatocyte cells, the human or animal hepatocarcinoma cell line group, dendritic cells, macrophage cells, Kuppfer cells and combinations thereof which may or may not be associated with lymphocytes. Advantageously, these cells are human hepatocarcinoma cells of the PLC/PRF/5 cell line.

Preferentially, the culture medium comprises, besides the ingredients required for culturing and the activating agent, at least one apoptosis-modulating agent. This apoptosis-modulating agent is chosen from interferons, anti-interferons, in particular anti-alpha and beta interferons; anti-caspases 3, in particular peptide analogs, such as z-VADfmk, i.e. N-benzyloxycarbonyl-Val-Ala-Asp-fluoromethyl ketone, and antibodies directed against anti-caspases 3.

In fact, the present inventors have observed that, in patients infected both with HIV and with Hepacivirus HCV, taking certain HIV protease inhibitors, such as Ritonavir (commercial name), causes an increase in the HCV virema associated with hepatic cytolysis. The observation of hepatic cytolysis immediately after taking a medicinal product which, in addition to its antiprotease activity, also modifies the regulation of apoptosis, is suggestive of induction of the signal for apoptosis. For this reason, it is advantageous to add to the method of the invention at least one apoptosis-modulating agent.

The suitable medium selected is in particular chosen from DMEM medium, or a medium derived from DMEM medium, and RPMI medium, or a medium derived from RPMI medium. Preferably, it is a medium derived from DMEM medium which comprises the complete DMEM medium marketed by the company Gibco BRL, supplemented with from 0 to 10 mM of sodium pyruvate, 0 to 10% of nonessential amino acids, 1 to 10 mM of glutamine, 100 to 200 U/ml of penicillin, 100 to 200 mg/ml of streptomycin and 1 to 20% of calf serum.

This medium advantageously also comprises 0.1 to 0.5% of BSA (bovine serum albumin) or of HSA (human serum albumin) coupled to a fatty acid, according to the method of Dixon et al., 1991, Journal of Biological Chemistry, vol. 226, p. 5080.

In a preferred embodiment of the method of the invention, prior to bringing the permissive cells into contact with the LVP fraction, they are washed in a PBS buffer preheated to a temperature of approximately 37° C.

The permissible cells thus infected under the conditions described above are subcultured several times and the presence of said virus is demonstrated in the infected permissible cells and in the culture supernatant by RT-PCR and/or by an immunological technique, such as indirect immunofluorescence using an antibody specific to said virus, and/or by flow cytometry.

The method for culturing, propagating and replicating described above is particularly suitable for culturing, propagating and replicating the viruses belonging to the Flaviviridae family and to the Hepacivirus genus, in particular the hepatitis C and hepatitis G viruses, including the GBV-A, GBV-B and GBV-C viruses, for which, to date, no effective culturing method existed.

The subject of the invention is also a culture medium for culturing viruses belonging to the Togaviridae and Flaviviridae families, which medium comprises the complete DMEM medium marketed by the company Gibco BRL, supplemented with 0 to 10 mM of sodium pyruvate, 0 to 10% of nonessential amino acids, 1 to 10 mM of glutamine, 100 to 200 U/ml of penicillin, 100 to 200 mg/ml of streptomycin and 1 to 20% of calf serum. This medium advantageously also comprises 0.1 to 0.5% of BSA (bovine serum albumin) or of HSA (human serum albumin) coupled to a fatty acid, according to the method given by way of reference above.

A subject of the invention is also a cell line infected with at least one virus belonging to the Togaviridae and Flaviviridae families, in which the cells in the cell line are permissible cells which have an endocytosis pathway relayed by at least one receptor for lipoproteins, which can be modulated, inter alia, by an activating agent, said cells being capable of propagating and replicating the virus. Preferentially, the receptor is the LSR and/or the receptor for LDLs, and advantageously the permissive cells have both receptors.

In particular, the cell line is a line in which the cells are derived by infecting a cell chosen from primary human or animal hepatocyte cells, cells of the human and animal hepatocarcinoma cell line group, in particular a cell originating from the human hepatocarcinoma cell line PLC/PRF/5 which has a surface receptor for lipoproteins which can be activated by fatty acids, denditric cells, macrophage cells and Kuppfer cells. However, the invention is not limited to a cell of a cell line which has at least the LSR surface receptor for lipoproteins, and encompasses the transfected or transformed cells which are capable of expressing, at their surface, the LSR receptor and/or the receptor for LDLs. The infected cell line obtained according to the method of the invention can be used for screening and/or selecting at least one antiviral molecule, according to which the antiviral molecule and the infected cell line are brought into contact.

The invention also relates to a method for preparing a composition for detecting, in a sample, antibodies directed against at least one virus belonging to the Togaviridae and Flaviviridae families, which comprises at least one partial or total purification of the viral particles of said virus or of the polypeptides obtained using one of the methods as defined above. In particular, said viral particles or said polypeptides are attached to a solid support.

Moreover, the invention relates to a method for obtaining antibodies or antibody fragments directed against at least one virus belonging to the Togaviridae and Flaviviridae families, according to which an animal is immunized with viral particles or polypeptides obtained using one of the methods as defined above. The production of polyclonal and monoclonal antibodies or of antibody fragments is part of the general knowledge of those skilled in the art. By way of example, mention may be made of Köhler G. and Milstein C. (1975), Continuous culture of fused cells secreting antibody of predefined specificity, Nature, 256: 495-497 and Galfre G. et al. (1977) Nature, 266: 522-550 for the production of polyclonal antibodies, and Roda A., Bolelli G. F. Production of high titer antibody to bile acids, Journal of Steroid Biochemistry, Vol. 13, pp 449-454 (1980), for the production of polyclonal antibodies. Antibodies may be produced immunizing mice or rabbits with the viral particles or the polypeptides obtained according to the method of the invention. For the production of monoclonal antibodies, the immunogen may be coupled to keyhole limpet hemocyanin (KLH peptide) as a support for immunization, or to serum albumin (SA peptide). The animals are given an injection of immunogen using complete Freund's adjuvant. The sera and the hybridoma culture supernatants derived from the immunized animals are analyzed for their specificity and their selectivity using conventional techniques, such as for example ELISA or Western Blot assays. The hybridomas which produce the most specific and the most sensitive antibodies are selected. Monoclonal antibodies may also be produced in vitro by cell culture of the hybridomas produced or by recovery of ascites fluid, after intraperitoneal injection of the hybridomas into mice. Whatever the method of production, by supernatant or by ascites, the antibodies are then purified. The purification methods used are essential ion exchange gel filtration and exclusion chromatography or immunoprecipitation. A sufficient number of antibodies are screened in functional assays in order to identify the most effective antibodies. The in vitro production of antibodies, of antibody fragments or of antibody derivatives, such as chimeric antibodies produced by genetic engineering, is well known to those skilled in the art.

More particularly, the term “antibody fragment” is intended to mean the F(ab)2, Fab, Fab′, sFv (Blazar et al., 1997, Journal of Immunology 159: 5821-5833 and Bird et al., 1988, Science 242: 423-426) of a native antibody, and the term “derivative” is intended to mean, inter alia, a chimeric derivative of a native antibody (see, for example Arakawa et al., 1996, J. Biochem 120: 657-662 and Chaudray et al., 1989, Nature 339: 394-397).

The invention also relates to a diagnostic composition comprising at least the viral particles of the polypeptides obtained according to the method of the invention, or the antibodies obtained according to the method defined above, and a diagnostic kit comprising, inter alia, said composition, and also an immunization composition comprising at least the viral particles or the polypeptides optionally associated with a pharmaceutically acceptable vehicle and/or excipient and/or adjuvant. However, the invention also opens up other therapeutic perspectives in that it makes possible to develop a therapeutic composition capable of qualitatively and/or quantitatively influencing the propagation and replication of the above-mentioned viruses in vivo, since it comprises, inter alia, a ligand capable of modulating, of repressing or of inhibiting the endocytosis pathway relayed by at least one of the receptors for lipoproteins, the ligand being chosen from an antagonistic antibody directed against said receptor, the production of which is within the scope of those skilled in the art, as described above, and an unnatural protein, i.e. a soluble protein obtained by genetic recombination, or a soluble synthetic polypeptide, which binds to said receptor, or in that it comprises, inter alia, at least one molecule which modulates, represses or inhibits the expression of the gene encoding said receptor or the activity of the promoter which encodes said receptor.

Finally, the subject of the invention is a method for screening and/or selecting at least one antiviral molecule, according to which said antiviral molecule and an infected cell line obtained according to one of the methods of the invention are brought into contact.

The term “virus belonging to the Togaviridae and Flaviviridae families”, as used in the invention, refers to any viral species, among which are strains pathogenic for humans, variant strains, attenuated strains and defective strains derived from said strains. In fact, RNA viruses are known to have a high spontaneous mutation rate. Multiple strains may, therefore, exist which may be more or less virulent. It is within the scope of those skilled in the art to identify such strains, for example by homology of nucleic acid and/or peptide sequences compared to a reference strain, and/or by identifying a strain or an isolate with respect to morphological and/or immunological criteria.

The term “cell line” refers to a culture of permissive cells in which the virus propagates after a first culturing, and therefore comprises, but is not limited to, the individual cells, the harvested cells and the cultures containing the cells in so far as they are derived from cells of the reference cell line. It is known that spontaneous or induced changes may occur in the caryotype during storage or transfer. Therefore, the cells derived from the reference cell line may not be strictly identical to the cells or cultures of origin, and the “cell line” also refers to the variants. The term “cell line” also includes immortalized cells.

The term “permissive cells” refers to all cells which are capable of propagating the virus, i.e. cells which have an endocytosis pathway relayed by at least one receptor for lipoproteins.

An immunization composition is a composition which comprises at least the viral particles or the polypeptides obtained according to the method of the invention, but is not limited to them, and also covers the recombinant proteins and fragments thereof which may be obtained using techniques of genetic recombination in a suitable host cell. Such an immunization composition may comprise, if necessary, a vehicle and/or an excipient and/or an adjuvant, on the condition that they are pharmaceutically acceptable.

The term “pharmaceutically acceptable vehicle” is intended to mean the supports and vehicles which can be administered to humans or to animals, as described for example in Remington's Pharmaceutical Sciences 16th Ed., Mack Publishing Co. The pharmaceutically acceptable vehicle is preferably isotonic or hypotonic or exhibits weak hypertonicity and a relatively low ionic strength. The definitions of the pharmaceutically acceptable excipients and adjuvants are also given in Remington's Pharmaceutical Sciences, mentioned above.

### EXAMPLE 1

### Preparation of the Biological Material

The lipoproteins are separated from the plasma or from the serum of a patient who has not eaten for 12 hours and who has been detected as positive for the hepatitis C virus. 1% of an EDTA solution (0.15 M NaCl-0.1 M EDTA) is added to the blood taken from the patient. The mixture is centrifuged for 10 minutes at 3500 rpm, at the temperature of 4° C. The plasma is then harvested and stored at 4° C. until used.

The patient's blood is harvested onto a dry tube and, after clotting, centrifuged for 10 minutes at 3000 rpm, at a temperature of 4° C. The serum is removed and stored at 4° C. until used.

(i) Production of a fraction comprising LVPs with a density of less than 1.0063 g/ml, of a fraction comprising LVPs with a density of between 1.0063 g/ml and 1.063 g/ml, and of a fraction comprising LVPs of greater than 1.063 g/ml.

The plasma and the serum are, respectively, ultracentrifuged for 4 hours at 100,000 rpm, at 4° C. in a TL100 machine marketed by the company Beckman and comprising a TL100.4 rotor. The upper fraction which contains the LVPs with a density of less than 1.0063 g/ml is recovered and stored at 4° C. The lower fraction is adjusted to a density of 1.063 g/ml by adding 7.21 g of NaBr per 100 ml of the fraction. The lower fraction is then ultracentrifuged for 4 hours at 100,000 rpm, at 4° C., in a TL100 machine comprising a TL100.4 rotor. The resulting upper fraction containing the fraction with a density of between 1.0063 g/ml and 1.063 g/ml is recovered and stored at 4° C.

(ii) Reduction of a fraction comprising LVPs with a density of less than 1.025 g/ml, of a fraction comprising LVPs with a density of between 1.025 g/ml and 1.063 g/ml, and of a fraction comprising LVPs with a density of greater than 1.063 g/ml.

The plasma and the serum are, respectively, adjusted to a final density of 1.025 g/ml by adding 2.518 g of NaBr per 100 ml. Centrifugation is carried out for 4 hours at 100,000 rpm, at 4° C., on the TL100 machine comprising a TL100.4 rotor.

The upper fraction which contains the fraction with a density of less than 1.025 g/ml is recovered and stored at 4° C. The lower fraction is just adjusted to a density of 1.063 g/ml by adding 4.84 g of NaBr per 100 ml. The lower fraction is then ultracentrifuged for 4 hours at 100,000 rpm, at 4° C., in the TL100 machine comprising a TL100.4 rotor. The resulting upper fraction containing the LDLs is recovered and stored at 4° C.

The various fractions harvested are then dialyzed for 18 hours at 4° C. against a 0.15 M NaCl/0.24 mM EDTA buffer. The fractions are then recovered and filtered over a 0.45μ membrane. A protein assay carried out by the Lowry method (Sigma).

### EXAMPLE 2

### Cell Culture

The culture medium used is DMEM medium (marketed by Gibco BRL) supplemented with: 1 mM of sodium pyruvate (Gibco), 1% of nonessential amino acids (Boehringer Mannheim), 2 mM of glutamine (Gibco BRL), 200 U/ml of penicillin (bioMérieux), 200 mg/ml of streptomycin (bioMérieux) and 10% of calf serum (Boehringer).

The cells originate from the PLC/PRF/5 cell line (Alexander cells) (ATCC reference: CRL 8024), which is a line established from a human hepatocarcinoma. This line, which has integrated a defective hepatitis B genome, secretes the HBs antigen of the hepatitis B virus but no infectious virus. The cells are cultured in the complete DMEM medium (Gibco-BRL) supplemented with 10% of foetal calf serum, mentioned above. The cells are washed with PBS (Gibco-BRL) preheated to 37° C. 1 ml of DMEM supplemented with 0.2% of BSA (Sigma) and 50 μg/ml of lipoproteins originating from each fraction or 100 μl of serum filtered over a 0.45μ membrane are added. Simultaneously, a solution of oleic acid in isopropanol, prepared beforehand (100 mM), is added in a concentration range of 0.1 to 0.8 mM. The cells are then subjected to incubation for 3 hours at 37° C. under a 5% CO₂ atmosphere. The medium is then replaced with complete DMEM medium supplemented with 10% of foetal calf serum. The cells are returned to the incubator under the conditions described above. The culture supernatants are regularly removed in order to serve, by RT-PCR, for the genome of the hepatitis C virus. In parallel, an analysis by immunofluorescence is carried out on the infected cells and controlled using an anti-HCV structural protein monoclonal antibody.

### EXAMPLE 3

### Results

The study is carried out using various LVP fractions obtained from plasma of two patients infected with HCV. The presence of the HCV genome in these various fractions was demonstrated by semi-nested RT-PCR.

| | LVP fractions with a | 1st | 2nd |
|---|---|---|---|
| Patient | density d*: | round | round |
| Patient No. 1 | d < 1.0063 | + | + |
| | 1.063 < d > 1.0063 | − | − |
| | d > 1.063 | − | + |
| Patient No. 2 | d < 1.0063 | − | + |
| | 1.063 < d > 1.0063 | − | + |
| | d > 1.063 | + | + |

*: density d in g/ml

These results show the presence of a signal positive for the presence of the HCV genome in the fractions with a density of less than 1.0063 g/ml, in both patients, either in the first round of PCR, in the second round of PCR.

The analysis of the fraction with a density of between 1.063 and 1.0063 g/ml is negative for the presence of the HCV genome in patient number 1, but positive in the second round of PCR in patient number 2. The fraction with a density of greater than 1.063 g/ml is positive for the presence of the HCV genome in both patients, but the response is weaker for patient 1 than for patient 2. These differences between patient no. 1 and patient no. 2 in the fraction with a density of between 1.063 and 1.0063 g/ml may be explained by a lower viral load in patient no. 1.

The results were obtained using culture supernatants after infection of 100 μl of serum taken from patients no. 1 and no. 2 in the presence or absence of 0.5 mM oleic acid. Negative controls were performed under the same conditions. The analysis performed by semi-nested RT-PCT shows that, for both patients, the presence of the HCV genome in the culture supernatants is detected at a higher frequency in the assays for which the infection was carried out in the presence of oleate.

Similar assays, carried out using the fractions with different densities (less than 1.0063 g/ml, between 1.0063 g/ml and 1.063 g/ml and greater than 1.063 g/ml), confirm these results and show that oleic acid facilitates the pathway for endocytosis of the LVPs at the time of the infection.

### EXAMPLE 4

### Study of the Kinetics of Attachment of the LVPs to the LSR Receptor of Hepatocytes in the Presence or Absence of Oleate

A fraction containing LVPs with a density of between 1.025 g/ml and 1.055 g/ml was obtained from an HCV-positive serum as described in example 1.

The amount of proteins present in the LVP fraction was determined by the Lowry method using the “Protein Assay” kit (Sigma Diagnostics) and the viral titer was evaluated by quantification of the HCV RNA by RT-PCR and real-time fluorescence detection (LightCycler™, Roche) (Wittwer et al., Biotechniques, 22: 176-181 (1997)).

The PLC/PRF/5 cell line, described in example 2 and cultured in DMEM medium-10% SVF, was used for studying the attachment of LVPs to the LSR receptor in the presence or absence of oleate.

The results were obtained according to the following protocol:

The PLC/PRF/5 cells were seeded at 1 million cells/well, so as to obtain, after incubation for 24 hours at 37° C., 5% CO₂, approximately 80% confluence.

Two washes in cold 1×PBS buffer were carried out, and then 1 ml of cold complete DMEM medium containing 0.2% of BSA is added to the PLC cells maintained on a bed of ice.

The LVP fraction (i.e. 10 mg of proteins and 1.7×10⁶ of copies of HCV RNA) is added to the cells in the presence or absence of 0.5 mM oleate. All the assays were carried out in triplicate.

An incubation was carried out at +4° C. for a period of time ranging from 1 to 9 hours. The cells were then washed with three times with cold 1×PBS, followed by lysees with 0.5 ml of lysis buffer from the Rneasy kit (Qiagen). The RNAs were then purified using this same kit and analyzed by quantitative RT-PCR (LightCycler™).

The results obtained are indicated in the attached figure, in which the incubation time is represented on the X-axis and the number of HCV RNA copies is represented on the Y-axis. In the graphic representation of this figure, the dark bars refer to a culture without oleate and the light bars refer to a culture with oleate. The results show that, in the presence of oleate, attachment of the LVPs to the LSR receptor is significantly increased compared with the assays without oleate.

### EXAMPLE 5

### Demonstration of the Association of the LVPs with Human Immunoglobulins

Various fractions containing LVPs harvested from plasma of three patients infected with HCV and prepared according to the protocol of example 1 were analyzed by polyacrylamide gel electrophoresis (PAGE) in the presence of SDS (sodium dodecyl sulfate) (SDS-PAGE) (Laemmli, Nature (1970), 227: 680-685). The presence of immunoglobulins (Igs) in these fractions was demonstrated using the Western Blot technique (Towbin et al., PNAS, (1979) 76: 4350-4354), using a peroxidase-coupled anti-human immunoglobulin goat serum (Jackson ImmunOResearch Laboratories, France). The results show that the human immunoglobulins are always detected in the fractions containing LVPs, in different amounts depending on the patients.

The amount of the HCV genome in these LVP fractions was measured by quantification of the HCV RNA by RT-PCT (RT=reverse transcriptase; PCRE=polymerase chain reaction) and real-time fluorescence detection (LIGHTCYCLER™, ROCHE) (Wittwer et al., Biotechniques (1997), 22: 176-181). The results show that the HCV RNA is always associated with the fractions containing LVPs, in different amounts depending on the patients.

The Ig associated LVPs (LVP/Ig+) were, in addition, purified using protein A coupled to beads, of the MAGmol Protein A MicroBeads type (Miltenyi Biotec, France) after passage through MS+ separation columns (Miltenyi Biotec, France), or using protein A-sepharose CL-4B (Pharmacia Biotech, France). In this case, all or most of the HCV RNA copurified with the Igs, as illustrated in the tables which follow. Consequently, using a fraction rich in LVPs, the samples used for the infections may be purified via their Igs so as to use preferentially LVP/Ig+/RNA+s.

Patient No. 1

| | d* < 1.0063 | d* 1.063 <d> 1.0063 |
|---|---|---|
| Presence of Igs | +++ | +++ |
| Quantification of | 27300 copies | 33600 copies |
| RNAs (per 0.2 ml of | | |
| LVPs) | | |
| Quantification of | 23625 copies | 31875 copies |
| RNAs copurified with | (86.5%) | (94.8%) |
| Ig | | |

d* signifies the density of the LVPs in g/ml.

Patient No. 2

| | d* < 1.0063 | d* 1.063 <d> 1.0063 |
|---|---|---|
| Presence of Igs | +++ | +/− |
| Quantification of | 32400 copies | 235800 copies |
| RNAs (per 0.2 ml of | | |
| LVPs) | | |
| Quantification of | 21300 copies | 21300 copies |
| RNAs copurified with | (65.7%) | (9%) |
| Ig | | |

d* signifies the density of the LVPs in g/ml.

Patient No. 3

| | d* < 1.0063 | d* 1.025 <d> 1.0055 |
|---|---|---|
| Presence of Igs | +++ | + |
| Quantification of | 197100 copies | 45900 copies |
| RNAs (per 0.2 ml of | | |
| LVPs) | | |
| Quantification of | 142500 copies | 26100 copies |
| RNAs copurified with | (72.3%) | (56.8%) |
| Ig | | |

d* signifies the density of the LVPs in g/ml.

These results show that, when immunoglobulins are present in the fractions containing. LVPs, the viral RNAs are mainly found in the fractions of LVPs associated with human immunoglobulins.

1-23. (Canceled)

24. A method for culturing, propagating and replicating, in vitro, the hepatitis C virus or the hepatitis G virus, according to which there is at least one lipo-viro-particles (LVP) fraction obtained from serum or from plasma of a patient infected with at least one virus selected from the group consisting of the hepatitis C virus and the hepatitis G virus, and said fraction is brought into contact with permissive cells having at least one receptor for lipoproteins, for a predetermined period of time in a suitable culture medium containing an activating agent selected from the group consisting of an unsaturated fatty acid and a derivative of an unsaturated fatty acid, said fatty acid comprising from 16 to 20 carbon atoms or a mixture thereof.

25. A method for culturing, propagating and replicating, in vitro, the hepatitis C virus or the hepatitis G virus, according to which there is at least one lipo-viro-particles (LVP) fraction, associated with human immunoglobulins, obtained from serum or from plasma of a patient infected with at least one virus selected from the group consisting of the hepatitis C virus and the hepatitis G virus, and said fraction is brought into contact with permissive cells having at least one receptor for lipoproteins, for a predetermined period of time in a suitable culture medium containing an activating agent selected from the group consisting of an unsaturated fatty acid and a derivative of an unsaturated fatty acid, said fatty acid comprising from 16 to 20 carbon atoms or a mixture thereof.

26. The method as claimed in claim 24, in which the receptor for lipoproteins is the lipolysis-stimulated receptor and/or the surface receptor for low density lipoproteins.

27. The method as claimed in claim 24, in which the unsaturated fatty acid is selected from the group consisting of oleic acid, palmitoleic acid, linoleic acid, linolenic acid, arachidonic acid, transhexadecenoic acid and elaidic acid, or derivatives thereof.

28. The method as claimed in claim 27, in which the fatty acid is oleic acid, which is added to said culture medium at a concentration of between 0.1 and 1 mM.

29. The method as claimed in claim 27, in which the fatty acid is oleic acid, which is added to said culture medium at a concentration of 0.5 mM.

30. The method as claimed in claim 24, in which the permissive cells are selected from the group consisting of primary human or animal hepatocyte cells, cells chosen from the human or animal hepatocarcinoma cell line group, dendritric cells, macrophage cells, Kuppfer cells and combinations thereof which may or may not be associated with lymphocytes.

31. The method as claimed in claim 30, in which the permissive cells are human hepatocarcinoma cells of the PLC/PRF/5 cell line.

32. The method as claimed in claim 24, in which the culture medium comprises, besides the ingredients required for culturing and the fatty acid or the derivative of the fatty acid, an apoptosis-modulating agent.

33. The method as claimed in claim 32, in which the apoptosis-modulating agent is selected from the group consisting of interferons, anti-interferons and anti-caspases 3.

34. The method as claimed in claim 24, in which the medium is DMEM medium, or a medium derived from DMEM medium, RPMI medium or a derivative of RPMI medium.

35. The method as claimed in claim 34, in which the medium is DMEM medium supplemented with 0 to 10 mM of sodium pyruvate, 0 to 10% of nonessential amino acids, 1 to 10 mM of glutamine, 100 to 200 U/ml of penicillin, 100 to 200 mg/ml of streptomycin and 1 to 20% of calf serum.

36. The method as claimed in claim 35, in which the medium is supplemented with 0.1 to 0.5% of BSA or with 0.1 to 0.5% of HSA coupled to a fatty acid.

37. The method as claimed in claim 24, in which, after bringing the permissive cells and said LVP fraction into contact, said permissive cells thus infected are subcultured several times and the presence of said virus is demonstrated in the permissive cells by RT-PCR and/or by an immunological technique.

38. A method for obtaining antibodies or antibody fragments directed against a virus selected from the group consisting of hepatitis C virus and hepatitis G virus, according to which an animal is immunized with viral particles or polypeptides obtained using a culturing method as claimed in claim 24.

39. A diagnostic composition comprising at least the antibodies obtained according to the method defined in claim 38.

40. A diagnostic kit also comprising a composition as defined in claim 39.

41. A method for screening and/or selecting at least one antiviral molecule, according to which infected permissive cells are obtained in accordance with claim 24 and said antiviral molecule is brought into contact with said infected permissive cells.

42. A method for preparing a composition for detecting, in a sample, antibodies directed against at least one virus selected from the group consisting of the hepatitis C virus and the hepatitis G virus, which comprises either (1) a purification of serum or plasma wherein at least one lipo-viro-particules fraction associated with human immunoglobulins is purified by using protein A or (2) a purification of the polypeptides obtained by using a culturing method as claimed in claim 25.

43. The method as claimed in claim 42, in which said viral particles or said polypeptides are attached to a solid support.

44. A diagnostic composition comprising at least the viral particles or the polypeptides obtained according to the method defined in claim 42.

45. A diagnostic kit also comprising a composition as defined in claim 44.

46. An immunization composition comprising at least the viral particles or the polypeptides obtained according to the method defined in claim 42, associated with a pharmaceutically acceptable vehicle and/or excipient and/or adjuvant.
